(19) United States
(12) Patent Application Publication
(10) Pub. No.: US 2020/0093786 A1
(43) Pub. Date: Mar. 26, 2020

(54) SYNERGISTIC USE OF CANNABIS FOR TREATING MULTIPLE MYELOMA

(71) Applicant: One World Cannabis LTD, Ramat Gan (IL)

(72) Inventors: Alon Sinai, Ramat Gan (IL); Ziv Turner, Kfar Saba (IL); Yehuda Baruch, Gedera (IL)

(21) Appl. No.: 16/559,826

(22) Filed: Sep. 4, 2019

Related U.S. Application Data
(62) Division of application No. 15/531,047, filed on May 26, 2017, now abandoned.
(60) Provisional application No. 62/084,568, filed on Nov. 26, 2014.

(51) Int. Cl. A61K 31/352; A61K 9/20; A61K 47/10; A61K 31/704; A61K 31/69; A61K 31/573; A61K 31/454; A61K 31/198; A61K 9/10; A61P 35/00; A61K 31/05; A61K 45/06; A61K 36/185

(52) U.S. Cl. CPC A61K 31/352; A61K 9/2009; A61K 9/2068; A61K 47/10; A61K 31/704; A61K 31/69; A61K 31/573; A61K 31/454; A61K 31/198; A61K 9/2095; A61K 9/2013; A61K 9/10; A61P 35/00; A61K 31/05; A61K 45/06; A61K 36/185; A61K 9/2059; A61K 9/2054; A61K 9/2027; A61K 9/2018; A61K 9/2077

(57) ABSTRACT

The present invention discloses a pharmaceutical composition comprising therapeutically effective amount of, or an extract consisting essentially therapeutically effective amount of at least one cannabinoid selected from the group consisting of: Cannabidiol (CBD) or a derivative thereof, Tetrahydrocannabinol (THC) or a derivative thereof, and any combination thereof, for use in the treatment of multiple myeloma (MM). The present invention further discloses methods and uses of the aforementioned composition.

### CROSS-REFERENCE TO RELATED APPLICATIONS

The present application is a divisional of U.S. application Ser. No. 15/531,047, filed Nov. 24, 2015, which claims benefit of U.S. Provisional Application No. 62/084,568, filed Nov. 26, 2014, the entire contents of both of which being hereby incorporated herein by reference.

### FIELD OF THE INVENTION

The present invention relates to a method and composition for treating Multiple Myeloma (MA) comprising at least one cannabinoid. More specifically, the present invention pertains to a method and composition comprising the cannabinoids Tetrahydrocannabinol (THC) and/or Cannabidiol (CBD).

### BACKGROUND OF THE INVENTION

Multiple myeloma (MM), also known as plasma cell myeloma, myelomatosis, or Kahler's, is a cancer of plasma cells, a type of white blood cell normally responsible for producing antibodies in which collections of abnormal plasma cells accumulate in the bone marrow, where they interfere with the production of normal blood cells. It is the second most common hematologic cancer as it accounts for 10% of all hematologic malignancies and represents 1% of all cancer diagnoses and 2% of all cancer deaths [1].

MM is the malignant disease which most frequently leads to bone lesions. Approximately 80% of myeloma patients develop osteoporosis, lytic bone lesions or fractures during the course of the disease. Of these patients 43% suffer pathological fractures most often of the vertebrae followed by fractures of the long bones [2].

Bone pain affects almost 70% of MM patients and is the most common symptom. Myeloma bone pain usually involves the spine and ribs, and worsens with activity. Persistent localized pain may indicate a pathological bone fracture. Involvement of the vertebrae may lead to spinal cord compression. Myeloma bone disease is due to the overexpression of Receptor Activator for Nuclear Factor κ B Ligand (RANKL) by bone marrow stroma. RANKL activates osteoclasts, which resorb bone. The resultant bone lesions are lytic in nature). The breakdown of bone also leads to release of calcium into the blood, leading to hypercalcemia and its associated symptoms.

MM is also commonly characterized in acute or chronic renal failure. The most common cause of renal failure is due to proteins secreted by the malignant cells. Myeloma cells produce monoclonal proteins of varying types, most commonly immunoglobulins and free light chains, resulting in abnormally high levels of these proteins in the blood. Depending on the size of these proteins, they may be excreted through the kidneys. Kidneys can be damaged by the tubulopathic effects of proteins or light chains. Increased bone resorption leads to hypercalcemia and causes nephrocalcinosis thereby also contributing to the renal failure. Amyloidosis is a distant third in the causation. Patients with Amyloidosis have high levels of Amyloid protein that can be excreted through the kidneys and cause damage to the kidneys and other organs. Other causes of renal failure in MM include hyperuricemia, recurrent infections and local infiltration of tumor cells.

MM treatments utilizing alkylating agents, corticosteroids, proteasome inhibitors, and immunomodulatory drugs have resulted in significant survival benefits, however relapse is inevitable and the disease remains incurable with a median survival of 5 years [3, 4].

Cannabinoids have been shown to inhibit the growth and induce apoptosis of a broad spectrum of tumor cells [5]. So far, two cannabinoid-specific receptors, CB₁ and CB₂, have been characterized from mammalian tissues [6]. They have been shown to possess anti-proliferative and anti-angiogenic effects in vitro as well as in vivo in different cancer models, Both cannabinoid systems are unambiguously osteo-protective, especially with regard to the aging skeleton, CB2 is expressed in osteoblasts and osteoclasts, stimulates bone formation, and inhibits bone resorption. Recently it has been discovered that CB2 receptor is highly expressed in MM cell lines [7]. Moreover, Cannabidiol (CBD) by itself or in synergy with bortezomib, strongly inhibited growth, arrested cell cycle progression and induced MM cells death by regulating the ERK, AKT and NF-KB pathways [8].

Several patent applications recite compositions for treating myeloma which involves substrates of the cannabinoid-specific receptors. For example, patent application US patent app. No. 20130172388 recites Novel CB2 inverse agonists for treating multiple myeloma and osteoporosis bone diseases and Patent application WO2014057067 discloses the use of a combination of endocannabinoids and cannabinoids complexes with a lipoprotein for the treatment of cancers dependent on hedgehog mechanisms of which MM is amongst them. The phsychotropic effect of these compositions is not yet known.

It is therefore a long felt and unmet need to formulate novel therapeutic anti-MM agents.

### SUMMARY OF THE INVENTION

It is thus one object of the present invention to disclose a pharmaceutical composition, wherein the composition comprises a therapeutically effective amount of Cannabidiol (CBD) or a derivative thereof and Tetrahydrocannabinol (THC) or a derivative thereof; in a predefined ratio, for use in the treatment of multiple myeloma (MM).

It is also an object of the present invention to disclose the aforementioned composition, wherein the CBD and the THC are in a predefined ratio conferring inhibition of multiple myeloma (MM) cells.

It is a further object of the present invention to disclose the pharmaceutical composition as defined above, wherein the CBD and the THC are in a predefined ratio conferring an additive effect with respect to inhibition of multiple myeloma (MM) cells relative to the effect conferred by the CBD and the THC administered separately in a similar concentration.

It is a further object of the present invention to disclose the pharmaceutical composition as defined in any of the above, wherein the CBD and the THC are in a predefined ratio conferring a synergistic effect with respect to inhibition of multiple myeloma (MM) cells relative to the effect conferred by the CBD and the THC administered separately in a similar concentration.

It is a further object of the present invention to disclose the pharmaceutical composition as defined in any of the above, wherein the predefined ratio of the CBD and the THC is about 1:1.

It is a further object of the present invention to disclose the pharmaceutical composition as defined in any of the above, wherein the predefined ratio of the CBD and the THC is about 1:5, respectively.

It is a further object of the present invention to disclose the pharmaceutical composition as defined in any of the above, wherein the predefined ratio of the CBD and the THC is about 5:1, respectively.

It is a further object of the present invention to disclose the pharmaceutical composition as defined in any of the above, wherein the predefined ratio of the CBD and the THC is about 1:4, respectively.

It is a further object of the present invention to disclose the pharmaceutical composition as defined in any of the above, wherein the inhibition of multiple myeloma (MM) cells is defined as at least 50% inhibition of multiple myeloma (MM) cells in vitro.

It is a further object of the present invention to disclose the pharmaceutical composition as defined in any of the above, wherein the CBD and the THC have a combination index (CI) value lower than 1 indicating synergism.

It is a further object of the present invention to disclose the pharmaceutical composition as defined in any of the above, wherein the CBD and the THC have a combination index (CI) value of 1 indicating an additive effect.

It is a further object of the present invention to disclose the pharmaceutical composition as defined in any of the above, wherein the concentration of the CBD or the derivative thereof is in the range of about 2% (wt.) to about 20%. (wt.).

It is a further object of the present invention to disclose the pharmaceutical composition as defined in any of the above, wherein the concentration of the THC or the derivative thereof is in the range of about 2% (wt.) to about 20% (wt.).

It is a further object of the present invention to disclose the pharmaceutical composition as defined in any of the above, wherein the composition comprises cannabis oil.

It is a further object of the present invention to disclose the pharmaceutical composition as defined in any of the above, wherein the cannabis oil is in a concentration of about 2% (wt.) to about 25% (wt.).

It is a further object of the present invention to disclose the pharmaceutical composition as defined in any of the above, wherein the composition comprises at least one excipient selected from the group consisting of: a solvent, absorbent, a sweetener, a disintegrant, a thickener, a binder, a lubricant, a glidant, an antiadherant, a coating agent, flavours, colours, sorbents, preservatives and any combination thereof.

It is a further object of the present invention to disclose the pharmaceutical composition as defined in any of the above, wherein the solvent is ethanol.

It is a further object of the present invention to disclose the pharmaceutical composition as defined in any of the above, wherein the composition is free of a pharmaceutically acceptable emulsifying agent or surfactant.

It is a further object of the present invention to disclose the pharmaceutical composition as defined in any of the above, wherein the composition is formulated for an administration route selected from the group consisting of: intranasal, transdermal, intravenous, vaginal, sublingual, buccal, oral, and any combination thereof.

It is a further object of the present invention to disclose the pharmaceutical composition as defined in any of the above, wherein the composition is formulated in a sublingual dosage form.

It is a further object of the present invention to disclose the pharmaceutical composition as defined in any of the above, wherein the composition is formulated in a solid dosage form.

It is a further object of the present invention to disclose the pharmaceutical composition as defined in any of the above, wherein the composition is formulated in a dosage form selected from the group consisting of syrup, drops, tincture, tablet, capsule, strip, film, spray, lozenge, effervescent form, solution, emulsion, suspension, granules, powder, and any combination thereof.

It is a further object of the present invention to disclose the pharmaceutical composition as defined in any of the above, wherein the THC and the CBD are formulated for penetrating the mucosal barrier.

It is a further object of the present invention to disclose the pharmaceutical composition as defined in any of the above, wherein the composition is formulated for rapid disintegration upon administration.

It is a further object of the present invention to disclose the pharmaceutical composition as defined in any of the above, wherein the composition is administered in combination with an additional MM therapeutic agent.

It is a further object of the present invention to disclose the pharmaceutical composition as defined in any of the above, wherein the additional MM therapeutic agent is selected from the group consisting of alkylating agents, corticosteroids, proteasome inhibitors, immunomodulatory drugs, and any combination thereof.

It is a further object of the present invention to disclose the pharmaceutical composition as defined in any of the above, wherein the additional MM therapeutic agent is selected from the group consisting of bortezomib (BTZ), lenalidomide (LEN), dexamethasone (DEX), melphalan (MEL), mitoxantrone, doxorubicin, Bortezomib-cyclophosphamide-dexamethasone (VCD), bortezomib-thalidomide-dexamethasone (VTD) and any combination thereof.

It is a further object of the present invention to disclose the pharmaceutical composition as defined in any of the above, wherein the composition confers inhibition of conventional chemotherapy resistant multiple myeloma (MM) cells.

It is a further object of the present invention to disclose the pharmaceutical composition as defined in any of the above, wherein the conventional chemotherapy comprises a MM therapeutic agent selected from the group consisting of bortezomib (BTZ), lenalidomide (LEN), mitoxantrone, dexamethasone (DEX), melphalan (MEL), doxorubicin (DOXO), Bortezomib-cyclophosphamide-dexamethasone (VCD), bortezomib-thalidomide-dexamethasone (VTD) and any combination thereof.

It is a further object of the present invention to disclose the pharmaceutical composition as defined in any of the above, wherein the composition is formulated in a sustained release dosage form or in a rapid release dosage form or in a combination thereof.

It is a further object of the present invention to disclose the pharmaceutical composition as defined in any of the above, wherein the sustained release dosage form is selected from the group consisting of liposomes, drug polymer conjugates, microencapsulation, controlled-release tablet coating, and any combination thereof.

It is a further object of the present invention to disclose the pharmaceutical composition as defined in any of the above, wherein the composition is not significantly psychoactive.

It is a further object of the present invention to disclose the pharmaceutical composition as defined in any of the above, wherein the composition is administered once, twice, three or four times through the day.

It is a further object of the present invention to disclose the pharmaceutical composition as defined in any of the above, wherein the THC or the CBD or both is derived from at least one cannabis plant.

It is a further object of the present invention to disclose the pharmaceutical composition as defined in any of the above, wherein the cannabis plant is a CBD rich strain.

It is a further object of the present invention to disclose the pharmaceutical composition as defined in any of the above, wherein the CBD rich strain is selected from a group consisting of Avidekel, Fedora 17, ACDC, and any combination thereof.

It is a further object of the present invention to disclose the pharmaceutical composition as defined in any of the above, wherein the cannabis plant is a THC rich strain.

It is a further object of the present invention to disclose the pharmaceutical composition as defined in any of the above, wherein the THC rich strain is selected from a group consisting of Black Destroyer, Critical Neville Haze, Mataro Blue, LSD OG Kush, Pineapple Chunk, Blue Monster Holk, Y Griega, Satori, Tutankhamon, and any combination thereof.

It is a further object of the present invention to disclose the pharmaceutical composition as defined in any of the above, wherein the CBD or derivative thereof is produced by a synthetic route.

It is a further object of the present invention to disclose the pharmaceutical composition as defined in any of the above, wherein the THC or derivative thereof is produced by a synthetic route.

It is a further object of the present invention to disclose the pharmaceutical composition as defined in any of the above, wherein the composition is dissolved in a lipophilic solvent or suspension carrier.

It is a further object of the present invention to disclose the pharmaceutical composition as defined in any of the above, wherein the lipophilic solvent or suspension carrier are selected from a group consisting of ethanol, medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, lecithin, vegetable oil, and any combination thereof.

It is a further object of the present invention to disclose a synergistically effective pharmaceutical composition, wherein the composition comprising a therapeutically effective amount of Cannabidiol (CBD) or a derivative thereof and Tetrahydrocannabinol (THC) or a derivative thereof in a predefined ratio conferring a synergistic effect with respect to inhibition of multiple myeloma (MM) cells, relative to the effect of the CBD and the THC administered separately in a similar concentration.

It is a further object of the present invention to disclose the synergistically effective pharmaceutical composition as defined above, wherein the predefined ratio of the CBD and the THC is selected from the group consisting of: about 1:1, 5:1, 1:5, 1:4 respectively.

It is a further object of the present invention to disclose a method of personalizing a cannabis dose regime to a patient with multiple myeloma (MM) comprising steps of: (a) monitoring cytotoxic effect of different THC:CBD ratios on MM cells isolated from the patient; and (b) providing the patient with a therapeutically effective cannabis dose regime comprising THC:CBD ratio selected according to step a.

It is a further object of the present invention to disclose a method of treating multiple myeloma (MM) in a subject; the method comprising steps of: (a) providing a composition according to claim 1; and (b) administrating the composition to the subject in a therapeutically effective dosage to treat MM is the subject.

It is a further object of the present invention to disclose the method as defined above, additionally comprising step of providing the CBD and the THC in a predefined ratio of about 1:5 or 5:1 or 1:1 or 1:4 respectively.

It is a further object of the present invention to disclose the method as defined in any of the above, additionally comprising steps of administrating the composition with the CBD and the THC in a predefined ratio conferring a synergistic effect with respect to inhibition of multiple myeloma (MM) cells relative to the CBD and the THC when administered separately in a similar concentration.

It is a further object of the present invention to disclose the method as defined in any of the above, additionally comprising steps of providing the composition comprising CBD concentration in the range of about 2% (wt) to about 20% (wt.).

It is a further object of the present invention to disclose the method as defined in any of the above, additionally comprising steps of providing the composition comprising THC concentration in the range of about 2% (wt.) to about 20% (wt.).

It is a further object of the present invention to disclose the method as defined in any of the above, additionally comprising steps of administering the composition in a route selected from the group consisting of: intranasal, transdermal, intravenous, vaginal, sublingual, buccal, oral, and any combination thereof.

It is a further object of the present invention to disclose the method as defined in any of the above, additionally comprising steps of administering the composition orally in a formulation selected from the group of preparations consisting of syrup, drops, tincture, tablet, strip, film, lozenge, capsule, solution, emulsion, suspension, spray, granules, powder, effervescent form, and any combination thereof.

It is a further object of the present invention to disclose the method as defined in any of the above, additionally comprising steps of administering the composition over a time period of about 1 day to about 6 months.

It is a further object of the present invention to disclose the method as defined in any of the above, additionally comprising steps of administering the composition in a dosage of CBD of up to 400 mg per day, preferably in the range of about 2 mg to about 400 mg per day.

It is a further object of the present invention to disclose the method as defined in any of the above, additionally comprising steps of administering the composition in a dosage of THC of up to 400 mg per day, preferably in the range of about 10 mg to about 400 mg per day.

It is a further object of the present invention to disclose the method as defined in any of the above, additionally comprising steps of administering the composition once, twice, three or four times through the day.

It is a further object of the present invention to disclose the method as defined in any of the above, additionally comprising steps of administering the composition with an additional MM therapeutic agent.

It is a further object of the present invention to disclose the method as defined in any of the above, additionally comprising steps of selecting the additional MM therapeutic agent from the group consisting of bortezomib (BTZ), lenalidomide (LEN), dexamethasone (DEX), melphalan (MEL), mitoxantrone, doxorubicin, and any combination thereof.

It is a further object of the present invention to disclose the method as defined in any of the above, additionally comprising steps of formulating the composition with at least one excipient selected from the group consisting of: a solvent, absorbent, a sweetener, a disintegrant, a thickener, a binder, a lubricant, a glidant, an antiadherant, a coating agent, flavours, colours, sorbents, preservatives and any combination thereof.

It is a further object of the present invention to disclose the method as defined in any of the above, additionally comprising steps of formulating the composition in a sustained release dosage form or in a rapid release dosage form or in a combination thereof.

It is a further object of the present invention to disclose the method as defined in any of the above, additionally comprising steps of formulating the composition in a sustained release dosage form selected from the group consisting of liposomes, drug polymer conjugates, microencapsulation, controlled-release tablet coating, and any combination thereof.

It is a further object of the present invention to disclose the method as defined in any of the above, additionally comprising steps of administering the composition to the subject without causing a significant psychoactive effect.

It is a further object of the present invention to disclose the method as defined in any of the above, additionally comprising steps of administering the CBD with Tetrahydrocannabinol. (THC) in a concentration which is equal or less than 20% (wt.).

It is a further object of the present invention to disclose the method as defined in any of the above, additionally comprising steps of inhibiting conventional chemotherapy resistant multiple myeloma (MM) cells.

It is a further object of the present invention to disclose a method of treating multiple myeloma (MM) in a subject; the method comprising steps of administrating to the subject a therapeutically effective amount of Cannabidiol (CBD) or a derivative thereof and Tetrahydrocannabinol (THC) or a derivative thereof in a predefined ratio conferring a synergistic effect with respect to inhibition of multiple myeloma (MM) cells, relative to the effect of the CBD and the THC administered separately in a similar concentration.

It is a further object of the present invention to disclose the method as defined above, wherein the predefined ratio between the CBD and the THC is of about 1:5 or 5:1 or 1:1 or 1:4 respectively.

It is a further object of the present invention to disclose a use of a composition comprising a therapeutically effective amount of Cannabidiol (CBD) or a derivative thereof and Tetrahydrocannabinol (THC) or a derivative thereof, in a predefined ratio, in the manufacture of a medicament for treating multiple myeloma (MM) of a subject.

It is a further object of the present invention to disclose the use as defined above, additionally comprising steps of providing the composition with CBD concentration in the range of about 2% (wt.) to about 20% (wt.).

It is a further object of the present invention to disclose the use as defined in any of the above, additionally comprising steps of providing the extract with THC concentration in the range of about 2% (wt.) to about 20% (wt.).

It is a further object of the present invention to disclose the use as defined in any of the above, additionally comprising steps of administering the composition in a route selected from a group consisting of: intranasal, transdermal, intravenous, vaginal, sublingual, buccal, oral, and any combination thereof.

It is a further object of the present invention to disclose the use as defined in any of the above, additionally comprising steps of administering the composition orally in a formulation selected from a group of preparations consisting of syrup, drops, tincture, tablet, strip, film, capsule, lozenge, spray, solution, emulsion, suspension, granules, powder, effervescent form, and any combination thereof.

It is a further object of the present invention to disclose the use as defined in any of the above, additionally comprising steps of administering the composition over a time period of about 1 day to about 6 months.

It is a further object of the present invention to disclose the use as defined in any of the above, additionally comprising steps of administering the composition in a dosage of CBD of up to 400 mg per day, preferably in the range of about 2 mg to about 400 mg per day.

It is a further object of the present invention to disclose the use as defined in any of the above, additionally comprising steps of administering the composition in a dosage of THC of up to 400 mg per day, preferably in the range of about 10 mg to about 400 mg per day.

It is a further object of the present invention to disclose the use as defined in any of the above, additionally comprising steps of administering the composition once, twice, three or four times through the day.

It is a further object of the present invention to disclose the use as defined in any of the above, additionally comprising steps of administering the composition with an additional MM therapeutic agent.

It is a further object of the present invention to disclose the use as defined in any of the above, selecting the additional MM therapeutic agent from the group consisting of bortezomib (BTZ), lenalidomide (LEN), dexamethasone (DEX), melphalan (MEL), mitoxantrone, doxorubicin, and any combination thereof.

It is a further object of the present invention to disclose the use as defined in any of the above, additionally comprising steps of formulating the composition with an excipient selected from a group consisting of a solvent, absorbent, a sweetener, a disintegrant, a thickener, a binder, a lubricant, a glidant, an antiadherant, a coating agent, flavours, colours, sorbents, preservatives and any combination thereof.

It is a further object of the present invention to disclose the use as defined in any of the above, additionally comprising steps of formulating the composition in a sustained release dosage form or in a rapid release dosage form or in a combination thereof.

It is a further object of the present invention to disclose the use as defined in any of the above, additionally comprising steps of selecting the sustained release dosage form from the group consisting of liposomes, drug polymer conjugates, microencapsulation, controlled-release tablet coating, and any combination thereof.

It is a further object of the present invention to disclose the use as defined in any of the above, additionally comprising steps of administering the composition to the subject without causing a significant psychoactive effect.

It is a further object of the present invention to disclose the use as defined in any of the above, additionally comprising steps of administering the CBD with Tetrahydrocannabinol (THC) in a concentration which is equal or less than 20%.

It is a further object of the present invention to disclose the use as defined in any of the above, wherein the CBD and the THC administered in a predefined ratio conferring a synergistic effect with respect to inhibition of multiple myeloma (MM) cells relative to the CBD and the THC administered separately in a similar concentration.

It is a further object of the present invention to disclose the use as defined in any of the above, wherein the CBD and the THC are administered in a ratio of about 1:5 or 5:1 or 1:1 or 1:4, respectively.

It is a further object of the present invention to disclose the use as defined in any of the above, wherein the synergistic effect is defined as at least 50% inhibition of multiple myeloma (MM) cells in vitro.

It is a further object of the present invention to disclose the use as defined in any of the above, wherein the synergistic effect is defined as more than about 80% inhibition of multiple myeloma (MM) cells in vitro.

It is a further object of the present invention to disclose the use as defined in any of the above, wherein the CBD and the THC have a combination index (CI) value of less than 1 indicating synergism.

It is a further object of the present invention to disclose a pharmaceutical composition comprising a therapeutically effective amount of Cannabidiol (CBD) or a derivative thereof and Tetrahydrocannabinol (THC) or a derivative thereof, in a predefined ratio, for use in the treatment of multiple myeloma (MM), wherein the composition is prepared by steps of: (a) preparing a mixture comprising an effective amount of cannabis oil, by a wet granulation process; and, (b) formulating the mixture in a solid dosage form by direct compression.

It is a further object of the present invention to disclose the pharmaceutical composition prepared by steps as defined above, wherein the mixture is further prepared by steps of: (a) preparing a first mixture comprising the cannabis oil and a solvent; (b) preparing a second mixture comprising at least one pharmaceutically acceptable carrier or excipient selected from the group consisting of a sweetener, a disintegrant, a thickener and any combination thereof; and (c) adding the second mixture to the first mixture by mixing using a high shear granulator.

It is a further object of the present invention to disclose the pharmaceutical composition prepared by steps as defined in any of the above, wherein the composition is further prepared by steps of: preparing the first mixture comprising cannabis oil, absorbent, lubricant and binder.

It is a further object of the present invention to disclose the pharmaceutical composition prepared by steps as defined in any of the above, wherein the composition is further prepared by steps of: (a) drying the mixture of step c to LOD equal or less than 1%; and (b) mixing the dried mixture with at least one pharmaceutically acceptable carrier or excipient selected from the group consisting of: glidant, binder, sweetener, lubricant, disintegrant and any combination thereof.

### BRIEF DESCRIPTION OF THE FIGURES

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

FIG. 1 is illustrating a diagram representing evaluation of the effect of different THC and CBD combinations on the viability of MM cells, as an embodiment of the present invention;

FIG. 2 is a illustrating a graph representing RPMIS MM cell line survival (%) vs. concentration (μM) of CBD, THC and their combinations, as an embodiment of the present invention;

FIG. 3 is illustrating a graph representing the combinatorial effect of CBD with THC;

FIGS. 4A-C are illustrating graphs representing the cytotoxic effect of CBD, THC and their combinations on CD138+ cells isolated from bone marrow aspirate of individual MM patients 1 to 3, respectively;

FIG. 4D is illustrating a graph representing CBD and THC IC50 (μM) values of individual patients 1 to 3; and

FIGS. 5 A-E are illustrating graphs representing the cytotoxic effect of CBD, THC and their combinations on different resistant MM cells.

### DETAILED DESCRIPTION OF THE PREFERRED EMBODIMENTS

The essence of the present invention is to provide a composition for treating multiple myeloma (MM) comprising Cannabidiol (CBD) and/or Tetrahydrocannabinol (THC) or any extract thereof. More specifically, the present invention recites a composition comprising cannabis extracts.

The term “multiple myeloma” or “MM” refers hereinafter to a cancer of plasma cells. More specifically, it is a clonal B-lymphocyte malignancy, which is characterized by the accumulation of terminally differentiated antibody-producing cells in the bone marrow. In multiple myeloma, collections of abnormal plasma cells accumulate in the bone marrow, where they interfere with the production of normal blood cells. Most cases of multiple myeloma also feature the production of a paraprotein an abnormal antibody which can cause kidney problems. Bone lesions and hypercalcemia (high blood calcium levels) are also often encountered. MM is also known as plasma cell myeloma, myelomatosis, or Kahler's disease.

The term “multiple myeloma cells” or “MM cells” as used herein refers to cell lines (of abnormal plasma cells) derived from MM subjects.

The term “inhibition of multiple myeloma cells” or “inhibition of MM cells” as used herein refers to an anti-MM effect including decrease in survival rate of MM cells, cytotoxic effect on MM cells, tumor size reduction, reduced viability of MM cells, apoptosis, cell cycle arrest, cell signaling arrest, mitochondrial trans membrane potential arrest and ROS production arrest.

The term “Cannabidiol” or “CBD” refers hereinafter to one of at least 85 active cannabinoids identified in cannabis. Cannabidiol is a major phytocannabinoid, accounting for up to 40% of the plant's extract. CBD is considered to have a wider scope of medical applications than Tetrahydrocannabinol (THC). Cannabidiol has a very low affinity for CB1 and CB2 receptors but acts as an indirect antagonist of their agonists. CBD may potentiate THC's effects by increasing CB1 receptor density or through another CB1-related mechanism. It is also an inverse agonist of CB2 receptors. CBD possesses antiproliferative, pro-apoptotic effects and inhibits cancer cell migration, adhesion and invasion.

The term “Tetrahydrocannabinol” or “THC” refers hereinafter to the principal psychoactive constituent (or cannabinoid) of the cannabis plant. THC has a partial agonist activity at the cannabinoid receptor CB1 and the CB2 receptor.

The term “THC rich cannabis strain” refers hereinafter to a cannabis strain having 20% or more THC, More specifically the term relates but is not limited to the following strains: Black Destroyer, Critical Neville Haze, Mataro Blue, LSD OG Kush, Pineapple Chunk, Blue Monster Holk, Y Griega, Satori, Tutankhamon.

The term “CBD rich cannabis strain” refers hereinafter to a cannabis strain having 1% or more CBD. More specifically the term relates but is not limited to the following strains: Avidekel, Fedora 17, ACDC.

The term “Avidekel” refers hereinafter to a cannabis strain comprising 15.8% CBD and less than 1% THC which may be found in patent application US 2014/0259228.

The term “Fedora 17” refers hereinafter to a cannabis strain having a cannabinoid profile consistently around 1% CBD with THC less than 0.1%.

The term “ACDC” refers hereinafter to a cannabis strain having about 19% CBD and a THC/CBD ratio of about 1:20.

The term “cannabinoid receptor” refers hereinafter to a class of cell membrane receptors under the G protein-coupled receptor superfamily. There are currently two known subtypes of cannabinoid receptors, termed CB1 and CB2. The CB1 receptor is expressed mainly in the brain, but also in the lungs, liver and kidneys. The CB2 receptor is expressed mainly in the immune system and in hematopoietic cells.

The term “Cannabinoid receptor type 1 (CB1)” refers hereinafter to a G protein-coupled cannabinoid receptor located primarily in the central and peripheral nervous system. It is activated by the endocannabinoid neurotransmitters anandamide and 2-arachidonoyl glyceride (2-AG); by plant cannabinoids, such as the compound THC, an active ingredient of the psychoactive drug cannabis; and by synthetic analogues of THC.

The term “Cannabinoid receptor type 2 (CB2)” refers hereinafter to a G protein-coupled receptor from the cannabinoid receptor family that in humans is encoded by the CNR2 gene. It is closely related to the cannabinoid receptor type 1, which is largely responsible for the efficacy of endocannabinoid-mediated presynaptic-inhibition, the psychoactive properties of Tetrahydrocannabinol, the active agent in marijuana, and other phytocannabinoids (natural cannabinoids). The principal endogenous ligand for the CB2 receptor is 2-arachidonoylglycerol (2-AG).

The term “nonpsychoactive” refers hereinafter to products or compositions or elements or components of cannabis not significantly affecting the mind or mental processes.

The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids.

The term “sustained release dosage form” refers hereinafter to the release of a drug at a predetermined rate in order to maintain a constant drug concentration for a specific period of time with minimum side effects. This can be achieved through a variety of formulations, including liposomes and drug-polymer conjugates. Sustained release in the present invention also includes within its scope “modified”, “controlled”, “sustained”, “prolonged”, “extended” or “delayed” release of a drug.

The term “rapid release dosage form” or “immediate release dosage form” as used herein refers to a drug or active ingredient or a composition or formulation, which disintegrates rapidly and gets dissolved to release the medicaments. Immediate release may be provided for by way of an appropriate pharmaceutically acceptable diluent or carrier, which diluent or carrier does not prolong, to an appreciable extent, the rate of drug release and/or absorption.

The term “XTT cell proliferation kit” refers hereinafter to a colorimetric assay for analyzing the number of viable cells. The assay is based on the cleavage of the tetrazolium salt XTT in the presence of an electron-coupling reagent, producing a soluble formazan salt. This conversion only occurs in viable cells. Cells grown in a 96-well tissue culture plate are incubated with the XTT labeling mixture for 2-20 hours. After this incubation period, the formazan dye formed is quantitated using a scanning multi-well spectrophotometer (ELISA reader). The measured absorbance directly correlates to the number of viable cells.

The present invention provides a pharmaceutical composition comprising therapeutically effective amount of, or an extract consisting essentially therapeutically effective amount of at least one cannabinoid selected from the group consisting of: Cannabidiol (CBD) or a derivative thereof, Tetrahydrocannabinol (THC) or a derivative thereof, and any combination thereof, for use in the treatment of multiple myeloma (MM).

The present invention further provides a synergistically effective pharmaceutical composition, wherein said composition comprising a therapeutically effective amount of Cannabidiol (CBD) or a derivative thereof and Tetrahydrocannabinol (THC) or a derivative thereof in a predefined ratio conferring a synergistic effect with respect to inhibition of multiple myeloma (MM) cells, relative to the effect of said CBD and said THC administered separately in a similar concentration.

According to one aspect of the present invention, the Cannabidiol (CBD) or a derivative thereof and Tetrahydrocannabinol (THC) or a derivative thereof, of the composition of the present invention are acting as modulators of the endocannabinoid system activity (i.e. cannabinoid receptors such as CB1 and CB2). According to other aspects of the present invention, cannabinoids may cause alteration of the immune function, and induction of apoptosis in abnormal cells, while not affecting normal cells.

Without wishing to be bound by theory, the THC component of the composition of the present invention may function by enhancing the apoptotic impact of the CBD, while exerting antineoplastic and proapoptotic effects. It is further noted that a synergistic effect is provided by the use of both cannabinoids, namely THC and CBD, which is not achievable with either compound alone. According to a specific embodiment, a composition comprising predetermined ratio between the two cannabinoids is provided by the present invention to treat MM.

It is according to one embodiment, to provide a pharmaceutical composition comprising therapeutically effective amount of, or an extract consisting essentially therapeutically effective amount of at least one cannabinoid selected from the group consisting of: Cannabidiol (CBD) or a derivative thereof, Tetrahydrocannabinol (THC) or a derivative thereof, and any combination thereof, for use in the treatment of multiple myeloma (MM).

The present invention further provides a pharmaceutical composition characterized by an effective amount of at least one cannabinoid selected from the group consisting of: Cannabidiol (CBD) or a derivative thereof, Tetrahydrocannabinol (THC) or a derivative thereof and any combination thereof, said CBD and said THC are in a predefined ratio conferring a synergistic effect with respect to inhibition of multiple myeloma (MM) cells relative to CBD and THC administered separately in a similar concentration.

It is further within the scope to provide the pharmaceutical composition as defined in any of the above, wherein CBD and THC are in a predefined ratio of about 5:1 or 1:5 or 1;1, respectively. It is further within the scope to provide the pharmaceutical composition as defined in any of the above, wherein the concentration of the CBD is in the range of about 2% to about 20%.

It is further within the scope to provide the pharmaceutical composition as defined in any of the above, wherein the concentration of the THC or the derivative thereof is in the range of about 2% to about 20%.

It is further within the scope to provide the pharmaceutical composition as defined in any of the above, wherein the composition comprises a therapeutically effective amount of Cannabidiol (CBD) or a derivative thereof and Tetrahydrocannabinol (THC) or a derivative thereof, in a predefined ratio, for use in the treatment of multiple myeloma (MM).

It is further within the scope to provide the pharmaceutical composition as defined in any of the above, wherein the CBD and the THC are in a predefined ratio conferring inhibition of multiple myeloma (MM) cells.

It is further within the scope to provide the pharmaceutical composition as defined in any of the above, wherein the CBD and the THC are in a predefined ratio conferring an additive effect with respect to inhibition of multiple myeloma (MM) cells relative to the effect conferred by the CBD and the THC administered separately in a similar concentration.

It is further within the scope to provide the pharmaceutical composition as defined in any of the above, wherein the CBD and the THC are in a predefined ratio conferring a synergistic effect with respect to inhibition of multiple myeloma (MM) cells relative to the effect conferred by the CBD and the THC administered separately in a similar concentration.

It is further within the scope to provide the pharmaceutical composition as defined in any of the above, wherein the predefined ratio of the CBD and the THC is about 1:1.

It is further within the scope to provide the pharmaceutical composition as defined in any of the above, wherein the predefined ratio of the CBD and the THC is about 1:5, respectively.

It is further within the scope to provide the pharmaceutical composition as defined in any of the above, wherein the predefined ratio of the CBD and the THC is about 5:1, respectively.

It is further within the scope to provide the pharmaceutical composition as defined in any of the above, wherein the predefined ratio of the CBD and the THC is about 1:4, respectively.

It is further within the scope to provide the pharmaceutical composition as defined in any of the above, wherein the inhibition of multiple myeloma (MM) cells is defined as at least 50% inhibition of multiple myeloma (MM) cells in vitro.

It is further within the scope to provide the pharmaceutical composition as defined in any of the above, wherein the CBD and the THC have a combination index (CI) value lower than 1 indicating synergism.

It is further within the scope to provide the pharmaceutical composition as defined in any of the above, wherein the CBD and the THC have a combination index (CI) value of 1 indicating an additive effect.

It is further within the scope to provide the pharmaceutical composition as defined in any of the above, wherein the concentration of the CBD or the derivative thereof is in the range of about 2% (wt.) to about 20%. (wt.).

It is further within the scope to provide the pharmaceutical composition as defined in any of the above, wherein the concentration of the THC or the derivative thereof is in the range of about 2% (wt.) to about 20% (wt.).

It is further within the scope to provide the pharmaceutical composition as defined in any of the above, wherein the composition comprises cannabis oil.

It is further within the scope to provide the pharmaceutical composition as defined in any of the above, wherein the cannabis oil is in a concentration of about 2% (wt.) to about 25% (wt.).

It is further within the scope to provide the pharmaceutical composition as defined in any of the above, wherein the composition comprises at least one excipient selected from the group consisting of: a solvent, absorbent, a sweetener, a disintegrant, a thickener, a binder, a lubricant, a glidant, an antiadherant, a coating agent, flavours, colours, sorbents, preservatives and any combination thereof.

It is further within the scope to provide the pharmaceutical composition as defined in any of the above, wherein the solvent is ethanol.

It is further within the scope to provide the pharmaceutical composition as defined in any of the above, wherein the composition is free of a pharmaceutically acceptable emulsifying agent or surfactant.

It is further within the scope to provide the pharmaceutical composition as defined in any of the above, wherein the composition is formulated for an administration route selected from the group consisting of: intranasal, transdermal, intravenous, vaginal, sublingual, buccal, oral, and any combination thereof.

It is further within the scope to provide the pharmaceutical composition as defined in any of the above, wherein the composition is formulated in a sublingual dosage form.

It is further within the scope to provide the pharmaceutical composition as defined in any of the above, wherein the composition is formulated in a solid dosage form.

It is further within the scope to provide the pharmaceutical composition as defined in any of the above, wherein the composition is formulated in a dosage form selected from the group consisting of syrup, drops, tincture, tablet, capsule, strip, film, spray, lozenge, effervescent form, solution, emulsion, suspension, granules, powder, and any combination thereof.

It is further within the scope to provide the pharmaceutical composition as defined in any of the above, wherein the THC and the CBD are formulated for penetrating the mucosal barrier.

It is further within the scope to provide the pharmaceutical composition as defined in any of the above, wherein the composition is formulated for rapid disintegration upon administration.

It is further within the scope to provide the pharmaceutical composition as defined in any of the above, wherein the composition is administered in combination with an additional MM therapeutic agent.

It is further within the scope to provide the pharmaceutical composition as defined in any of the above, wherein the additional MM therapeutic agent is selected from the group consisting of alkylating agents, corticosteroids, proteasome inhibitors, immunomodulatory drugs, and any combination thereof.

It is further within the scope to provide the pharmaceutical composition as defined in any of the above, wherein the additional MM therapeutic agent is selected from the group consisting of bortezomib (BTZ), lenalidomide (LEN), dexamethasone (DEX), melphalan (MEL), mitoxantrone, doxorubicin, Bortezomib-cyclophosphamide-dexamethasone (VCD), bortezomib-thalidomide-dexamethasone (VTD) and any combination thereof.

It is further within the scope to provide the pharmaceutical composition as defined in any of the above, wherein the composition confers inhibition of conventional chemotherapy resistant multiple myeloma (MM) cells.

It is further within the scope to provide the pharmaceutical composition as defined in any of the above, wherein the conventional chemotherapy comprises a MM therapeutic agent selected from the group consisting of bortezomib (BTZ), lenalidomide (LEN), mitoxantrone, dexamethasone (DEX), melphalan (MEL), doxorubicin (DOXO), Bortezomib-cyclophosphamide-dexamethasone (VCD), bortezomib-thalidomide-dexamethasone (VTD) and any combination thereof.

It is further within the scope to provide the pharmaceutical composition as defined in any of the above, wherein the composition is formulated in a sustained release dosage form or in a rapid release dosage form or in a combination thereof.

It is further within the scope to provide the pharmaceutical composition as defined in any of the above, wherein the sustained release dosage form is selected from the group consisting of liposomes, drug polymer conjugates, microencapsulation, controlled-release tablet coating, and any combination thereof.

It is further within the scope to provide the pharmaceutical composition as defined in any of the above, wherein the composition is not significantly psychoactive.

It is further within the scope to provide the pharmaceutical composition as defined in any of the above, wherein the composition is administered once, twice, three or four times through the day.

It is further within the scope to provide the pharmaceutical composition as defined in any of the above, wherein the THC or the DID or both is derived from at least one cannabis plant.

It is further within the scope to provide the pharmaceutical composition as defined in any of the above, wherein the cannabis plant is a CBD rich strain.

It is further within the scope to provide the pharmaceutical composition as defined in any of the above, wherein the CBD rich strain is selected from a group consisting of Avidekel, Fedora 17, ACDC, and any combination thereof.

It is further within the scope to provide the pharmaceutical composition as defined in any of the above, wherein the cannabis plant is a THC rich strain.

It is further within the scope to provide the pharmaceutical composition as defined in any of the above, wherein the THC rich strain is selected from a group consisting of Black Destroyer, Critical Neville Haze, Mataro Blue, LSD OG Kush, Pineapple Chunk, Blue Monster Holk, Y Griega, Satori, Tutankhamon, and any combination thereof.

It is further within the scope to provide the pharmaceutical composition as defined in any of the above, wherein the CBD or derivative thereof is produced by a synthetic route.

It is further within the scope to provide the pharmaceutical composition as defined in any of the above, wherein the THC or derivative thereof is produced by a synthetic route.

It is further within the scope to provide the pharmaceutical composition as defined in any of the above, wherein the composition is dissolved in a lipophilic solvent or suspension carrier.

It is further within the scope to provide the pharmaceutical composition as defined in any of the above, wherein the lipophilic solvent or suspension carrier are selected from a group consisting of ethanol, medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, lecithin, vegetable oil, and any combination thereof.

It is further within the scope to provide a synergistically effective pharmaceutical composition, wherein the composition comprising a therapeutically effective amount of Cannabidiol (CBD) or a derivative thereof and Tetrahydrocannabinol (THC) or a derivative thereof in a predefined ratio conferring a synergistic effect with respect to inhibition of multiple myeloma (MM) cells, relative to the effect of the CBD and the THC administered separately in a similar concentration.

It is further within the scope to provide the synergistically effective pharmaceutical composition as defined in any of the above, wherein the predefined ratio of the CBD and the THC is selected from the group consisting of: about 1:1, 5:1, 1:5, 1:4 respectively.

It is further within the scope to provide a method of personalizing a cannabis dose regime to a patient with multiple myeloma (MM) comprising steps of: (a) monitoring cytotoxic effect of different THC:CBD ratios on MM cells isolated from the patient; and (b) providing the patient with a therapeutically effective cannabis dose regime comprising THC:CBD ratio selected according to step a.

It is further within the scope to provide a method of treating multiple myeloma (MM) in a subject; the method comprising steps of: (a) providing a composition according to claim 1; and (b) administrating the composition to the subject in a therapeutically effective dosage to treat MM is the subject.

It is further within the scope to provide the method as defined above, additionally comprising step of providing the CBD and the THC in a predefined ratio of about 1:5 or 5:1 or 1:1 or 1:4 respectively.

It is further within the scope to provide the method as defined in any of the above, additionally comprising steps of administrating the composition with the CBD and the THC in a predefined ratio conferring a synergistic effect with respect to inhibition of multiple myeloma (MM) cells relative to the CBD and the THC when administered separately in a similar concentration.

It is further within the scope to provide the method as defined in any of the above, additionally comprising steps of providing the composition comprising CBD concentration in the range of about 2% (wt) to about 20% (wt.).

It is further within the scope to provide the method as defined in any of the above, additionally comprising steps of providing the composition comprising THC concentration in the range of about 2% (wt.) to about 20% (wt.).

It is further within the scope to provide the method as defined in any of the above, additionally comprising steps of administering the composition in a route selected from the group consisting of: intranasal, transdermal, intravenous, vaginal, sublingual, buccal, oral, and any combination thereof.

It is further within the scope to provide the method as defined in any of the above, additionally comprising steps of administering the composition orally in a formulation selected from the group of preparations consisting of syrup, drops, tincture, tablet, strip, film, lozenge, capsule, solution, emulsion, suspension, spray, granules, powder, effervescent form, and any combination thereof.

It is further within the scope to provide the method as defined in any of the above, additionally comprising steps of administering the composition over a time period of about 1 day to about 6 months.

It is further within the scope to provide the method as defined in any of the above, additionally comprising steps of administering the composition in a dosage of CBD of up to 400 mg per day, preferably in the range of about 2 mg to about 400 mg per day.

It is further within the scope to provide the method as defined in any of the above, additionally comprising steps of administering the composition in a dosage of THC of up to 400 mg per day, preferably in the range of about 10 mg to about 400 mg per day.

It is further within the scope to provide the method as defined in any of the above, additionally comprising steps of administering the composition once, twice, three or four times through the day.

It is further within the scope to provide the method as defined in any of the above, additionally comprising steps of administering the composition with an additional MM therapeutic agent.

It is further within the scope to provide the method as defined in any of the above, additionally comprising steps of selecting the additional MM therapeutic agent from the group consisting of bortezomib (BTZ), lenalidomide (LEN), dexamethasone (DEX), melphalan (MEL), mitoxantrone, doxorubicin, and any combination thereof.

It is further within the scope to provide the method as defined in any of the above, additionally comprising steps of formulating the composition with at least one excipient selected from the group consisting of: a solvent, absorbent, a sweetener, a disintegrant, a thickener, a binder, a lubricant, a glidant, an antiadherant, a coating agent, flavours, colours, sorbents, preservatives and any combination thereof.

It is further within the scope to provide the method as defined in any of the above, additionally comprising steps of formulating the composition in a sustained release dosage form or in a rapid release dosage form or in a combination thereof.

It is further within the scope to provide the method as defined in any of the above, additionally comprising steps of formulating the composition in a sustained release dosage form selected from the group consisting of liposomes, drug polymer conjugates, microencapsulation, controlled-release tablet coating, and any combination thereof.

It is further within the scope to provide the method as defined in any of the above, additionally comprising steps of administering the composition to the subject without causing a significant psychoactive effect.

It is further within the scope to provide the method as defined in any of the above, additionally comprising steps of administering the CBD with Tetrahydrocannabinol (THC) in a concentration which is equal or less than 20% (wt).

It is further within the scope to provide the method as defined in any of the above, additionally comprising steps of inhibiting conventional chemotherapy resistant multiple myeloma (MM) cells.

It is further within the scope to provide a method of treating multiple myeloma (MM) in a subject; the method comprising steps of administrating to the subject a therapeutically effective amount of Cannabidiol (CBD) or a derivative thereof and Tetrahydrocannabinol (THC) or a derivative thereof in a predefined ratio conferring a synergistic effect with respect to inhibition of multiple myeloma (MM) cells, relative to the effect of the CBD and the THC administered separately in a similar concentration.

It is further within the scope to provide the method as defined in any of the above, wherein the predefined ratio between the CBD and the THC is of about 1:5 or 5:1 or 1:1 or 1:4 respectively.

It is further within the scope to disclose a use of a composition comprising a therapeutically effective amount of Cannabidiol (CBD) or a derivative thereof and Tetrahydrocannabinol (THC) or a derivative thereof, in a predefined ratio, in the manufacture of a medicament for treating multiple myeloma (MM) of a subject.

It is further within the scope to disclose the use as defined above, additionally comprising steps of providing the composition with CBD concentration in the range of about 2% (wt.) to about 20% (wt.).

It is further within the scope to disclose the use as defined in any of the above, additionally comprising steps of providing the extract with THC concentration in the range of about 2% (wt.) to about 20% (wt.).

It is further within the scope to disclose the use as defined in any of the above, additionally comprising steps of administering the composition in a route selected from a group consisting of: intranasal, transdermal, intravenous, vaginal, sublingual, buccal, oral, and any combination thereof.

It is further within the scope to disclose the use as defined in any of the above, additionally comprising steps of administering the composition orally in a formulation selected from a group of preparations consisting of syrup, drops, tincture, tablet, strip, film, capsule, lozenge, spray, solution, emulsion, suspension, granules, powder, effervescent form, and any combination thereof.

It is further within the scope to disclose the use as defined in any of the above, additionally comprising steps of administering the composition over a time period of about 1 day to about 6 months.

It is further within the scope to disclose the use as defined in any of the above, additionally comprising steps of administering the composition in a dosage of CBD of up to 400 mg per day, preferably in the range of about 2 mg to about 400 mg per day.

It is further within the scope to disclose the use as defined in any of the above, additionally comprising steps of administering the composition in a dosage of THC of up to 400 mg per day, preferably in the range of about 10 mg to about 400 mg per day.

It is further within the scope to disclose the use as defined in any of the above, additionally comprising steps of administering the composition once, twice, three or four times through the day.

It is further within the scope to disclose the use as defined in any of the above, additionally comprising steps of administering the composition with an additional MM therapeutic agent.

It is further within the scope to disclose the use as defined in any of the above, selecting the additional MM therapeutic agent from the group consisting of bortezomib (BTZ), lenalidomide (LEN), dexamethasone (DEX), melphalan (MEL), mitoxantrone, doxorubicin, and any combination thereof.

It is further within the scope to disclose the use as defined in any of the above, additionally comprising steps of formulating the composition with an excipient selected from a group consisting of a solvent, absorbent, a sweetener, a disintegrant, a thickener, a binder, a lubricant, a glidant, an antiadherant, a coating agent, flavours, colours, sorbents, preservatives and any combination thereof.

It is further within the scope to disclose the use as defined in any of the above, additionally comprising steps of formulating the composition in a sustained release dosage form or in a rapid release dosage form or in a combination thereof.

It is further within the scope to disclose the use as defined in any of the above, additionally comprising steps of selecting the sustained release dosage form from the group consisting of liposomes, drug polymer conjugates, microencapsulation, controlled-release tablet coating, and any combination thereof.

It is further within the scope to disclose the use as defined in any of the above, additionally comprising steps of administering the composition to the subject without causing a significant psychoactive effect.

It is further within the scope to disclose the use as defined in any of the above, additionally comprising steps of administering the CBD with Tetrahydrocannabinol (THC) in a concentration which is equal or less than 20%.

It is further within the scope to disclose the use as defined in any of the above, wherein the CBD and the THC administered in a predefined ratio conferring a synergistic effect with respect to inhibition of multiple myeloma (MM) cells relative to the CBD and the THC administered separately in a similar concentration.

It is further within the scope to disclose the use as defined in any of the above, wherein the CBD and the THC are administered in a ratio of about 1:5 or 5:1 or 1:1 or 1:4, respectively.

It is further within the scope to disclose the use as defined in any of the above, wherein the synergistic effect is defined as at least 50% inhibition of multiple myeloma (MM) cells in vitro.

It is further within the scope to disclose the use as defined in any of the above, wherein the synergistic effect is defined as more than about 80% inhibition of multiple myeloma NM) cells in vitro.

It is further within the scope to disclose the use as defined in any of the above, wherein the CBD and the THC have a combination index (CI) value of less than 1 indicating synergism.

It is further within the scope to disclose a pharmaceutical composition comprising a therapeutically effective amount of Cannabidiol (CBD) or a derivative thereof and Tetrahydrocannabinol (THC) or a derivative thereof, in a predefined ratio, for use in the treatment of multiple myeloma (MM), wherein the composition is prepared by steps of: (a) preparing a mixture comprising an effective amount of cannabis oil, by a wet granulation process; and, (b) formulating the mixture in a solid dosage form by direct compression.

It is further within the scope to disclose a pharmaceutical composition prepared by steps as defined above, wherein the mixture is further prepared by steps of: (a) preparing a first mixture comprising the cannabis oil and a solvent; (b) preparing a second mixture comprising at least one pharmaceutically acceptable carrier or excipient selected from the group consisting of a sweetener, a disintegrant, a thickener and any combination thereof; and (c) adding the second mixture to the first mixture by mixing using a high shear granulator.

It is further within the scope to disclose a pharmaceutical composition prepared by steps as defined in any of the above, wherein the composition is further prepared by steps of: preparing the first mixture comprising cannabis oil, absorbent, lubricant and binder.

It is further within the scope to disclose a pharmaceutical composition prepared by steps as defined in any of the above, wherein the composition is further prepared by steps of: (a) drying the mixture of step c to LOD equal or less than 1%; and (b) mixing the dried mixture with at least one pharmaceutically acceptable carrier or excipient selected from the group consisting of: glidant, binder, sweetener, lubricant, disintegrant and any combination thereof.

It is further within the scope to provide the pharmaceutical composition as defined in any of the above, wherein the composition is adapted to be administered in a route selected from a group consisting of: intranasal, transdermal, intravenous, oral, and any combination thereof.

It is further within the scope to provide the pharmaceutical composition as defined in any of the above, wherein the CBD or the derivative thereof interacts with at least one receptor selected from a group consisting of Cannabinoid receptor type 1 (CB1), Cannabinoid receptor type 2 (CB2), and any combination thereof.

It is further within the scope to provide the pharmaceutical composition as defined in any of the above, wherein the THC or the derivative thereof interacts with at least one receptor selected from a group consisting of Cannabinoid receptor type 1 (CB1), Cannabinoid receptor type 2 (032), and any combination thereof.

It is further within the scope to provide the pharmaceutical composition as defined in any of the above, wherein the composition additionally comprises inactive ingredients selected from a group consisting of antiadherants, binders, coatings, disintegrants, flavours, colourants, lubricants, glidants, sorbents, preservatives, sweeteners, and any combination thereof.

It is further within the scope to provide the pharmaceutical composition as defined in any of the above, wherein CBD and THC administered in a ratio of about 1:1, respectively confers a synergistic effect with respect to inhibition of multiple myeloma (MM) cells relative to the CB1) and the THC administered separately in a similar concentration.

It is further within the scope to provide the pharmaceutical composition as defined in any of the above, wherein CBD and THC administered in a ratio of about 1:5, respectively confers a synergistic effect with respect to inhibition of multiple myeloma (MM) cells relative to the CBD and the THC administered separately in a similar concentration.

It is further within the scope to provide the pharmaceutical composition as defined in any of the above, wherein CBD and THC administered in a ratio of about 5:1, respectively confers a synergistic effect with respect to inhibition of multiple myeloma (MM) cells relative to CBD and THC administered separately in a similar concentration.

It is further within the scope to provide the pharmaceutical composition as defined in any of the above, wherein CBD and THC administered in a ratio of about 1:4, respectively confers a synergistic effect with respect to inhibition of multiple myeloma (MM) cells relative to CBD and THC administered separately in a similar concentration.

It is further within the scope to provide the pharmaceutical composition as defined in any of the above, wherein the synergistic effect is defined as at least 50% inhibition on RPMI8226 multiple myeloma (MM) cells in vitro.

It is further within the scope to provide the pharmaceutical composition as defined in any of the above, wherein the synergistic effect is defined as more than about 80% inhibition on RPMI8226 multiple myeloma (MM) cells in vitro.

It is according to another embodiment, to provide a method of treating multiple myeloma (MM) in a subject; the method comprising administrating to the subject a therapeutically effective amount of, or an extract consisting essentially therapeutically effective amount of at least one cannabinoid selected from the group consisting of Cannabidiol (CBD) or a derivative thereof, Tetrahydrocannabinol (THC) or a derivative thereof, and any combination thereof.

It is according to another embodiment, to disclose the use of a composition comprising a therapeutically effective amount of, or an extract consisting essentially a therapeutically effective amount of at least one cannabinoid selected from the group consisting of: Cannabidiol (CBD) or a derivative thereof, Tetrahydrocannabinol (TEC) or a derivative thereof, and any combination thereof in the manufacture of a medicament to treat multiple myeloma (MM).

It is further within the scope to provide the use of a composition as defined in any of the above, wherein CBD and THC administered in a ratio of about 1:1, respectively confers a synergistic effect with respect to inhibition of multiple myeloma (MM) cells relative to CBD and THC administered separately in a similar concentration.

It is further within the scope to provide the use of a composition as defined in any of the above, wherein CBD and THC administered in a ratio of about 1:5, respectively confers a synergistic effect with respect to inhibition of multiple myeloma (MM) cells relative to CBD and THC administered separately in a similar concentration.

It is further within the scope to provide the use of a composition as defined in any of the above, wherein CBD and THC administered in a ratio of about 5:1, respectively confers a synergistic effect with respect to inhibition of multiple myeloma (MM) cells relative to CBD and THC administered separately in a similar concentration.

It is further within the scope to provide the use of a composition as defined in any of the above, wherein CBD and THC administered in a ratio of about 1:4, respectively confers a synergistic effect with respect to inhibition of multiple myeloma (MM) cells relative to CBD and THC administered separately in a similar concentration.

It is further within the scope to provide the use of a composition as defined in any of the above, wherein the synergistic effect is defined as at least 50% inhibition on RPMIS multiple myeloma (MM) cells in vitro.

It is further within the scope to provide the use of a composition as defined in any of the above, wherein the synergistic effect is defined as more than about 80% inhibition on RPMIS multiple myeloma (MM) cells in vitro.

It is further within the scope to provide the use of a composition as defined in any of the above, wherein CBD and THC have a combination index (CI) value of less than 1 indicating synergism.

In order to understand the invention and to see how it may be implemented in practice, a plurality of preferred embodiments will now be described, by way of non-limiting example only, with reference to the following examples.

### Example 1

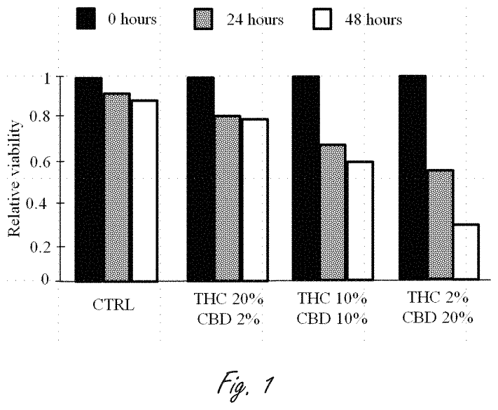

Reference is now made to FIG. 1 which demonstrates a graph of the relative viability of Multiple myeloma (MM) cells vs. different concentrations of CBD and THC, during different time periods (i.e. 0, 24 and 48 hours). The effect of different concentrations of CBD and THC on the viability of different multiple myeloma cell lines and primary cells isolated from bone marrow of myeloma patients in the presence and absence of bone marrow stroma cells, was tested. Several MM cell lines were plated at 2×10⁴ cells per well in 96-wells and reacted with different concentrations of CBD and THC. Samples were taken from bone marrow aspirates from MM patients. Mononuclear cells were separated by Ficoll density gradient centrifugation and myeloma cells were selected using CD138 microbeads (Miltenyi Biotec). Purified CD138+ patient cells were plated at a density of 2×10⁴ cells per well and treated for 48 hours with different concentrations of CBD and THC (THC 2% CBD 20%; THC 10% CBD 10%; and THC 20% CBD 2%). Cell viability was measured using XTT cell proliferation Kit (Biological Industries) according to manufacture instructions. It can be seen from FIG. 1, that in comparison to the control sample (in which only buffer was added), all combinations of CBD and THC showed an effect upon the viability of the cells.

### Example 2

Reference is now made to a study evaluating the anti-MM activity induced by the combination of CBD, THC; CBD:THC 1:1; 5:1 and 1:5 respectively with other MM chemotherapeutic drugs in vitro. It is herein acknowledged that combinations of novel and/or conventional anti-MM agents can achieve higher clinical response rates than single agent(s). In addition, many patients experience significant dose-limiting side effects requiring dose reductions or cessation of therapy. Therefore, the response of MM cells to CBD, THC; CBD:THC 1:1; 5:1 and 1:5 respectively in combination with currently in use anti-MM agents, such as (bortezomib (BTZ), lenalidomide (LEN), dexamethasone (DEX), melphalan (MEL) and doxorubicin (DOXO) was evaluated. The anti-MM activity of combined treatment was analyzed by XTT assays (i.e. as described in Example 1), and the presence of synergistic cytotoxic effects was evaluated using the Chou-Talalay method based on the median-effect equation and the classic isobologram equation and compusyn computer software.

It appears that in comparison to the control (in which only buffer was added), or to currently in use anti-MM agents, all combinations of CBD and THC affected the viability of the cells.

### Example 3

This example presents a study of the mode of action of cannabis as an anti-myeloma agent. The effect of cannabis on MM cell lines was evaluated on: apoptosis, cell cycle, mitochondrial trans membrane potential, ROS production, and cell signaling:

Apoptosis analysis: MM cells are treated with different concentrations of CBD, THC; CBD:THC 1:1; 5:1 and 1:5 respectively during different intervals of time. For evaluation of apoptosis, cells are processed using an Annexin V/propidium iodide (PI) kit (Becton Dickinson Biosciences) according to the manufacture instructions.

Cell-cycle analysis: MM cells are exposed to different concentrations of CBD, THC; CBD:THC 1:1; 5:1 and 1:5 respectively for different intervals of time, permeabilized by 70% ethanol at −20° C. overnight and incubated with 50 μg/ml PI and 20 units/ml RNase-A (Roche Diagnostics). DNA content is analyzed by flow cytometry. Data collection is performed using FACSCalibur (Becton Dickinson) and analysis is performed with the CellQuest software.

Cell signaling: MM cell lines are plated in RPMI 1640 with 10% FBS, penicillin, and streptomycin. CBD, THC; CBD:THC 1:1; 5:1 and 1:5 respectively are added for 0, 30 minutes and 2, 6, 24 and 48 h. Cells are lysed in RIPA-lysis buffer containing 10 mM sodium pyrophosphate, 2 mM sodium orthovanadate, 5 mM sodium fluoride, 5 g/mL aprotinin, 5 g/mL leupeptin, and 1 mM phenylmethylsulfonyl fluoride. Proteins are separated by sodium dodecyl sulfate-polyacrylamide gel electrophoresis, transferred onto nitrocellulose membranes and immunoblotted with cell signaling antibodies. Immunoreactive bands are detected by Western Blot chemiluminescence reagents (Thermo Scientific) and exposed on Kodak-XAR film.

Cell signaling arrest was achieved with the THC and CBD extract combinations, with different THC and CBD ratios providing different levels of arrest.

Mitochondrial transmembrane potential: Mitochondrial transmembrane potential (Dwm) is evaluated by 5,5′,6′6′-tetrachloro-1,1′,3,3′-tetraehylbenzimidazolylcarbocyanineiodide (JC-1) staining. Briefly, 2×10⁴ cells are treated with of CBD, THC; CBD:THC 1:1; 5:1 and 1:5 respectively for different times and then incubated for 10 min at room temperature with 10 ng/ml of JC-1. JC-1 is excited by an argon laser (488 nm), and the green (530 nm)/red (570 nm) emission fluorescence is collected simultaneously. Carbonyl cyanide chlorophenylhydrazone protonophore, a mitochondrial uncoupler that collapses (Dwm), is used as a positive control. Samples are analyzed using a FACScan cytofluorimeter with CellQuest software.

Different levels of reduction arrest in mitochondrial transmembrane potential were achieved with the various THC and CBD combinations of the present invention.

ROS production: The fluorescent probe dichlorodihydrotluorescein diacetate (DUDA) is used to assess oxidative stress levels. Briefly, 2×10⁴ cells treated with the appropriate compounds are incubated with 20 μM DCFA (Life Technologies Italia, Italy) 20 min. prior to the harvest time point. The cells are then washed, and the intensity of the fluorescence are assayed using flow cytometry and CellQuest software.

Different levels of reduction arrest ROS production was obtained with the THC and CBD extracts herein described.

### Example 4

This example presents the effect of cannabis on bone homeostasis. It is herein acknowledged that the crosstalk among the MM cells, osteoblasts (OBs) and osteoclasts (OCs) results in bone destruction [9-12]. To study the effect of cannabis on OB function, MC3T3-E1 pre-osteoblastic cells (ATCC) and bone marrow-derived stromal cells were cultured in osteoblastic differentiation media, with or without MM in the presence of different concentrations of CBD, THC; CBD:THC 1:1; 5:1 and 1:5 respectively for different periods of time. At the end of the culture period, cells were evaluated for OB differentiation. To evaluate the effect of cannabis on OC function, mononuclear cells from MM patients were differentiated to osteoclasts and treated with cannabis and their activity was evaluated in the presence and absence of stroma cells.

### Example 5

This example examines the anti-tumor efficacy of cannabis in murine xenograft MM model. SCID mice (6-8 week old) were maintained in accordance with Institutional Animal Care Use Committee guidelines. Mice were gamma-irradiated (150 rads) using Cs137 γ-irradiator source and (24 hrs post-irradiation) injected subcutaneously with MM cells (7×10⁶/mouse) suspended in PBS. 2-3 weeks later, when palpable tumors developed, mice were randomized into different groups (10 mice/group), and the following treatment protocol was implemented: Group 1: vehicle control was administered ip, every day, 5 days a week throughout the duration of the experiment; Group 2-4: the best combination(s) of CBD:THC 1:1; 5:1 and 1:5 according to in vitro results at different doses (1, 10 and 20 mg/kg) were administered ip, every day, 5 days a week throughout the duration of experiment; Group 5-6: THC and CBD at 20 mg/kg administered ip, every day, 5 days a week throughout the duration of experiment. The tumor is removed and analyzed at the end of the experiment. Evaluation of efficacy includes inhibition of tumor growth, survival, blood tests, animals' vital signs and gross pathology. Tumor size is measured by caliper. Caliper measurements of the longest perpendicular tumor diameters are performed every other day to estimate tumor volume. Glucose and oxytocin level is evaluated on peripheral blood. All compositions showed decrease in tumor size in a ratio dependent manner.

### Example 6

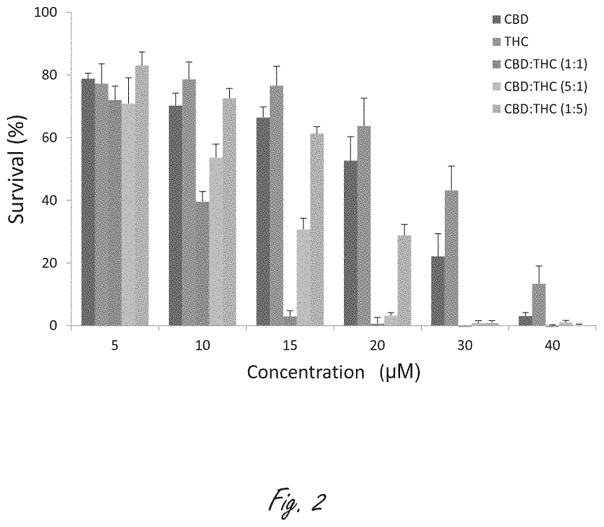

This example examines the cytotoxic effect of CBD alone, THC alone and combinations of both compounds. The cytotoxic effect of CBD, THC and their combinations in different ratios such as CBD:THC 1:1; CBD:THC 5:1 and CBD:THC 1:5 were evaluated on RPMI8226 multiple myeloma (MM) human cell lines. Reference is now made to FIG. 2 which presents a graph of RPMIS MM cell line survival (%) vs. concentration (μM).

As illustrated in FIG. 2, CBD and THC, and their combinations decreased the survival of MM cells in a concentration dependent manner. The dose that caused 50% of MM cell death was 16 μM and 22 μM for CBD and THC, respectively.

It is demonstrated in this figure that treatment with CBD in combination with THC had synergistic effects, the cytotoxic effect being higher with each of the three combinations tested, relative to treatment with CBD or THC separately.

Furthermore, in a concentration of about 15 μM and more, the cytotoxic effect of CBD and THC combinations (e.g. CBD:THC 1:1; CBD:THC 5:1) has demonstrated less than 30% survival of RPMIS MM cells, while treatment with CBD or THC separately demonstrated higher than about 70% survival rate of the RPMIS MM cells. Moreover, in a concentration of about 20 μM and higher, the cytotoxic effect of all CBD and THC combinations (i.e. CBD:THC 1:1; CBD:THC 5:1 and CBD:THC 5:1), demonstrated less than 30% survival of RPMIS MM cells, while treatment with CBD or THC separately gave about 50% survival rate of the RPMIS MM cells. Thus, this experiment demonstrates the significantly higher cytotoxic effect of CBD and THC combinations as compared to their effect when administered separately.

### Example 7

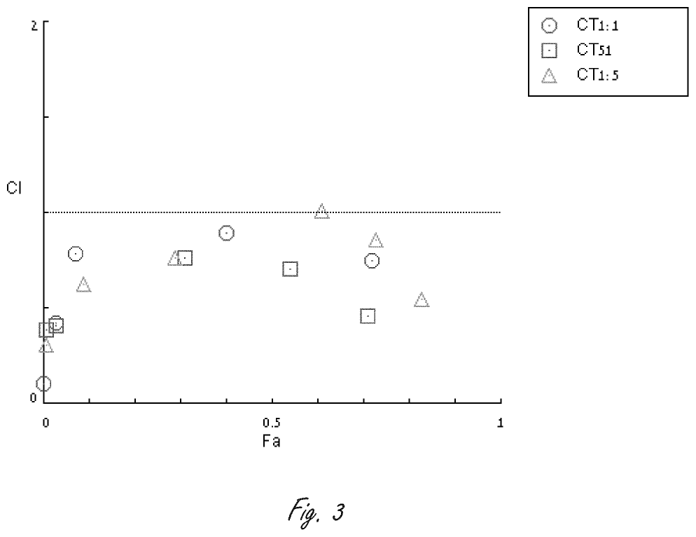

This experiment shows the combinatorial effect of CBD when administered together with THC. Reference is now made to FIG. 3 which presents a graph of the ratio of the THC and/or CBD fraction affected (Fa) vs. the Combination Index (CI). The graph demonstrates the effect of the combination of CBD with THC upon RPMI8226 MM cells. RPMIS cells were cultured for 48 hours with CBD and THC and compared to their combinations (i.e. CBD:THC 1:1; CBD:THC 5:1 and CBD:THC 1:5).

As illustrated in FIG. 3, the CI value <1, CI=1 and CI>1 indicates quantitative definition of synergism, additive effect, and antagonism, respectively. Each treatment was performed in triplicate in four independent experiments and presented as mean±SE.

It is shown that the combination of CBD and THC in the ratio of 1:1 is with CI less than 0.9. In another exemplary embodiment, the combination of CBD and THC in the ratio of 5:1 is with CI less than 0.7. The different ratios of the combination of CBD and THC (i.e. CBD:THC 1:1; CBD:THC 5:1 and CBD:THC 1:5) demonstrate CI<1 thereby, exhibiting synergy.

### Example 8

Cytotoxic Effect of CBD, THC and their Combinations

The aim of this example is to study the effect of CBD, THC, as compared to their combinations (CBD:THC 1:1; 5:1 and 1:5 respectively) on the viability of different multiple myeloma cell lines and primary cells isolated from bone marrow of myeloma patients in the presence and absence of bone marrow stroma cells.

Several MM cell lines were plated at 2×10⁴ cells per well in 96-wells and treated with different concentrations of CBD, THC and their combinations (CBD:THC 1:1; 5:1 and 1:5 respectively). For patient samples, bone marrow aspirates from MM patients were collected, and mononuclear cells were separated by Ficoll density gradient centrifugation and myeloma cells selected using CD138 microbeads (Miltenyi Biotec). Purified CD138⁺ patient cells were plated at a density of 2×10⁴ cells per well and treated for 48 h with different concentrations of CBD, THC; CBD:THC 1:1; 5:1 and 1:5 respectively. Peripheral blood samples from MM patients and healthy donors are processed by Ficoll density gradient centrifugation to isolate peripheral blood mononuclear cells (PBMCs). PBMCs are plated at 2×10⁴ cells per well and exposed to different concentrations of CBD, THC; CBD:THC 1:1; 5:1 and 1:5 respectively for 48 h. Cell viability is measured using XTT cell proliferation Kit (Biological Industries) according to the manufacture instructions. For co-culture assays MM cells are stained with CFSE, cultured in the presence of HS-5 human stroma cell line, treated with the drugs and their viability is evaluated by counterstained with PI and cell viability evaluation by flow cytometer analysis.

Reference is now made to an experiment demonstrating the cytotoxic effects of CBD, THC and their combinations (CBD:THC 1:1; 5:1 and 1:5 respectively) on CD138+ cells from myeloma patients.

CD138+ cells were isolated from bone marrow aspirate of MM patients and cultured during 48 hours with CBD, THC and their combination (CBD:THC 1:1; CBD:THC 5:1 and CBD:THC 1:5). XTT assay was performed to assess cell viability. Each treatment was performed in triplicate and presented as mean±SE.

Reference is now made to Table 1, presenting data on 3 MM patients tested in this experiment. In the table, SM refers to Smoldering Myeloma, M refers to Myeloma, VTD refers to Bortezomib-thalidomide-dexamethasone and VCD refers to bortezomib-cyclophosphamide-dexamethasone.

TABLE 1
Data on MM patients tested for the cytotoxic
effect of CBD, THC and their combinations

| Patient | Diagnose | Treatment | Age | Sex |
|---|---|---|---|---|
| 1 | SM | NO | 49 | F |
| 2 | M | VTD | 81 | M |
| 3 | M | VCD | 70 | F |

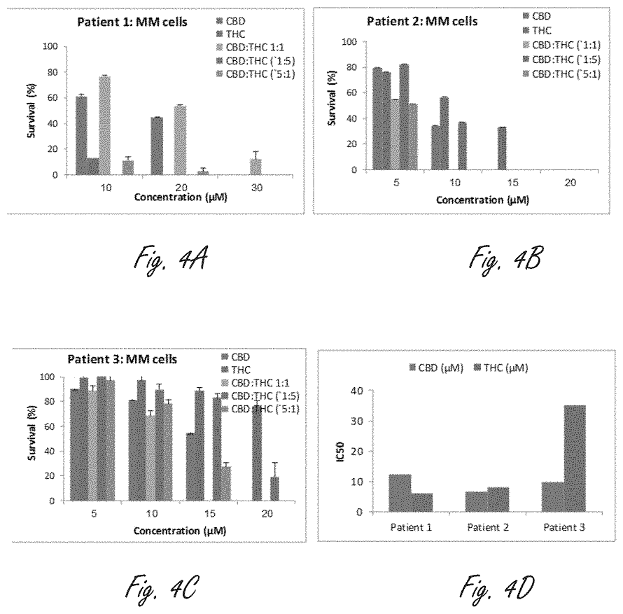

Reference is now made to FIG. 4, presenting the evaluation of the cytotoxic effect of CBD and THC as compared to their combinations (CBD:THC 1:1; CBD:THC 5:1 and CBD:THC 1:5) on multiple myeloma (MM) cells derived from three MM patients (described in table 1). FIG. 4 A-C graphically illustrating MM cells survival (%) vs. concentration. FIG. 4D graphically illustrates the IC50 dose (the dose that caused 50% MM cell death) for each of the 3 patients of table 1.

As can be seen in FIG. 4, CBD and THC decreased survival of MM cells in a concentration dependent manner in each of the patients tested. The dose that caused 50% of MM cell death (IC50) was 6.7-12.5 μM and 6-35 μM for CBD and THC, respectively (FIG. 4D).

The treatment with CBD in combination with THC had synergistic effect, with respect to survival of MM cells, in each of the three combinations tested (FIG. 4 A-C). There were differences in the sensitivity of the patients to the combinations:
Patient 1 was less sensitive to CBD than to THC. The combination which was more effective for this patient was CBD:THC 1:5.
Patient 2 was slightly more sensitive to CBD than to THC. The combinations which were more effective for this patient were CBD:THC 1:5 and CBD:THC 5:1.
Patient 3 was less sensitive to THC than to CBD. The combinations which were more effective for this patient were CBD:THC 1:1 and CBD:THC 5:1.

In view of the above results it can be concluded that MM patient culture cells are sensitive to CBD and THC treatment. The cytotoxic effect of CBD and THC combination is higher than the effect of each one of the cannabinoids alone.

It is further demonstrated that the CBD and THC combinations and formulations of the present invention can be designed in a patient specific manner. In other words, the THC and CBD combination ratios are customized for individual patients. In this personalized therapy model, medical decisions, practices, and/or products are being tailored to the individual patient. A diagnostic testing is often employed for selecting appropriate and optimal CBD and THC combination therapy based on the context of a patient's genetic content or other molecular or cellular analysis.

To test the combinatorial effect of CBD together with THC, CD138+ cells were isolated from bone marrow aspirate of MM patients and cultured during 48 hours with CBD, THC and their combination (CBD:THC 1:1; CBD:THC 5:1 and CBD:THC 1:5).

Reference is now made to Table 2 presenting combinatorial effect results of CBD with THC. Combination Index (CI) value <1, =1, >1 indicates synergism, additive effect, and antagonism, respectively. Pat 1, 2, 3 indicate the patient number. Each treatment was performed in triplicate in four independent experiments and presented as mean SE.

TABLE 2
Combinatorial effect of CBD with THC

| | CBD (μM) | THC (μM) | CI |
|---|---|---|---|
| CBD:THC 1:1 | | | |
| Pat 1 | 10.0 | 10.0 | 3.1 |
| | 30.0 | 30.0 | 5.0 |
| Pat 2 | 10.0 | 10.0 | 0.8 |
| | 20.0 | 20.0 | 1.6 |
| Pat 3 | 15.0 | 15.0 | 0.5 |
| | 20.0 | 20.0 | 0.6 |
| CBD:THC 1:5 | | | |
| Pat 1 | 1.9 | 10.0 | 0.4 |
| | 3.8 | 20.0 | 0.9 |
| Pat 2 | 2.8 | 15.0 | 0.6 |
| | 3.8 | 20.0 | 0.8 |
| Pat 3 | 1.9 | 10.0 | 1.0 |
| | 3.8 | 20.0 | 0.6 |
| CBD:THC 5:1 | | | |
| Pat 1 | 10.0 | 1.6 | 0.7 |
| | 30.0 | 4.9 | 1.0 |
| Pat 2 | 5.0 | 0.8 | 0.8 |
| | 10.0 | 1.6 | 0.6 |
| Pat 3 | 20.0 | 3.3 | 0.6 |
| | 30.0 | 4.9 | 0.9 |

It is clearly shown that, in most of the patients and concentrations tested, the cytotoxic effect of CBD and THC combinations on MM patient culture cells is more than additive or synergistic. The optimal CBD and THC ratio and concentration for obtaining the cytotoxic synergistic effect, is dependent upon the individual patient.

### Example 9

The Effect of CBD, THC and their Combination on Viability of MM Cells Regardless of Sensitivity to Conventional Chemotherapy

The cytotoxic effect of CBD and THC as compared to their combinations (CBD:THC 1:1; CBD:THC 5:1 and CBD:THC 1:5) was evaluated on MM cell lines resistant to anti-MM agents currently in use, such as RPMI-MR20 (mitoxantrone-resistant cells), RPMI-LR5 (LEN-resistant cells) and RPMI-Dox40 (DOXO-resistant cells) after 48 hours of treatment.

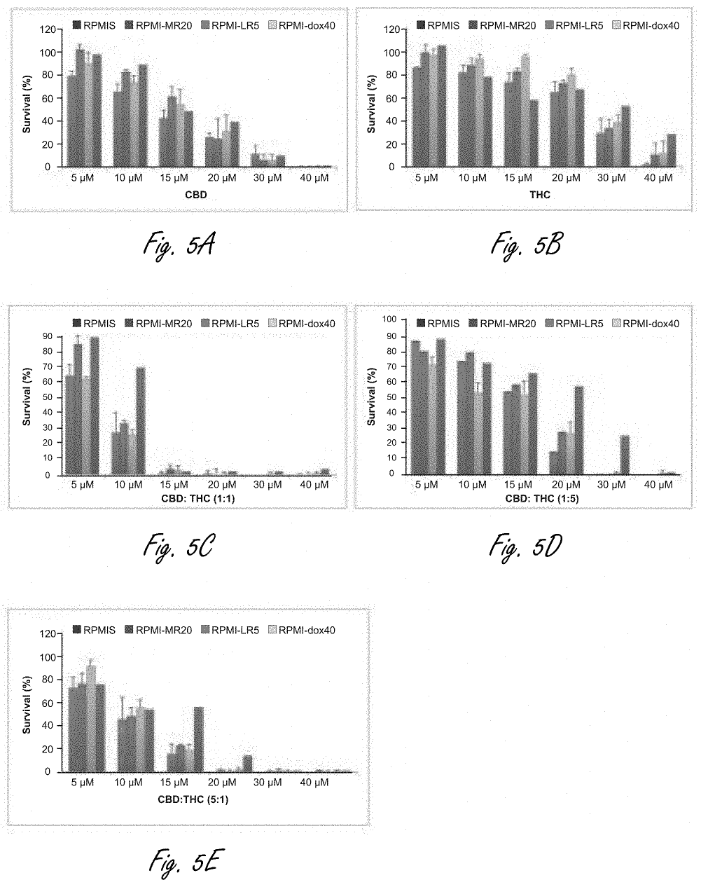

Reference is now made to FIG. 5 illustrating the cytotoxic effect of CBD, THC and their combinations on MM cells resistant to conventionally used anti-MM agents. RPMI-MR20, RPMI-LR5 and RPMI-Dox40 were cultured during 48 hours with CBD (FIG. 5A), THC (FIG. 5B), CBD:THC 1:1 (FIG. 5C), CBD:THC 1:5 (FIG. 5D) and CBD:THC 5:1 (FIG. 5E). XTT assay was performed to assess cell viability. Each treatment was performed in triplicate in three independent experiments and presented as mean±SE).

As demonstrated by the results described in FIG. 5, CBD and THC and their combinations decreased survival of MM cells in a concentration dependent manner regardless of the MM cells resistant to other conventionally used anti-MM. Thus, it can be concluded that CBD, THC and their combination reduce viability of MM cells regardless of sensitivity to conventional chemotherapy.

### Example 10

A Tablet Formulation Containing THC and CBD

Reference is now made to a process for producing a tablet with enhanced penetration of THC and CBD through oral administration. Using a wet granulation technology, cannabis oil is combined with dry powder components to produce a tablet with good hardness characteristics which disintegrates rapidly upon administration. The THC and CBD ingredients in the resultant tablet can penetrate the mucosal barrier without emulsification.

Reference is now made to Table 3 presenting ingredients and production process of a solid oral formulation containing cannabis oil to provide 10 mg of THC and 2.5 mg of CBD (40% of THC and 10% of CBD), as an embodiment of the present invention.

TABLE 3
A solid formulation containing THC and CBD combination

| Core | | Liquids | | |
|---|---|---|---|---|
| %/tablet | mg/tablet | mg/tablet | Raw Materials | Function |
| | | | PART I Wet | |
| | | | Granulation | |
| 12.5 | 25.00 | | Cannabis Oil | API |
| | | 20.0 | Ethanol | Solvent |
| 20 | 40.00 | | Mannitol | Sweetener |
| | | | | disintegrant |
| 12.5 | 25.00 | | Plasdone K25 | Thickener |
| | | | PART II Direct | |
| | | | Compression | |
| 20.5 | 41.00 | | Corn Starch | Disintegrant |
| | | | | binder |
| 25 | 50.00 | | Mannitol | Sweetener |
| | | | | disintegrant |
| 1.5 | 3.00 | | Magnesium Stearate | Lubricant |
| 5.0 | 10.00 | | Aerosil 200 | Glidant |
| | | | (Silicone Dioxide) | |
| 3.0 | 6.00 | | Croscarmellose Sodium | Disintegrant |
| 100 | 200 | | Total | |

Reference is now made to manufacturing steps of the solid formulation comprising THC and CBD combinations:

1. Slowly adding Ethanol to Cannabis oil throughout an intensive mixing and observing liquid homogeneity. A

2. Mixing Mannitol and Plasdone 25 (Polymer of 1-vinyl-2-pyrrolidone) in a blender. B

3. Slowly adding B to A with mixing (use high-shear granulator). C

4. Drying C at 80° C. until LOD less than 1%. D

5. Milling D and sieving with 120 micron screen sieve. E

6. Mixing in a blender E with Corn Starch, Mannitol, Magnesium Stearate, Silica and Croscarmelose Sodium. F

7. Compressing tablets with a tableting press machine.

It is within the scope that a solid formulation containing THC and CBD combinations as described above has cytotoxic effect on MM cells and may be efficacious for treating MM patients.

Reference is now made to a formulation and a manufacturing process of a hydrophobic tablet matrix, for hydrophobic cannabis oil, as a further example of the composition and process of the present invention.

For the production of the hydrophobic tablet matrix a wet granulation process is applied, during which, ethanolic solution of cannabis oil is absorbed by a mix of Aerosil 972 and carnauba wax. After the steps of drying and milling, a green granulate is obtained. At the step of direct compression, mannitol, hypromellose and silica are added to improve the blend flowability. Addition of hydrophobic components is optional.

Table 4 exemplifies ingredients and process of a hydrophobic tablet matrix containing cannabis oil.

TABLE 4
A hydrophobic tablet matrix containing
THC and CBD combination

| Core | | Liquids | | |
|---|---|---|---|---|
| %/tablet | mg/tablet | mg/tablet | Raw Materials | Function |
| | | | PART I Wet | |
| | | | Granulation | |
| 20.00% | 50.00 | | Cannabis Oil | API |
| | | | (20% THC/5% CBD) | |
| | | 10.0 | Ethanol | Solvent |
| 16.00% | 40.00 | | Hydrophobic | Absorbent |
| | | | fumed silica | |
| | | | (Aerosil 972) | |
| 8.00% | 20.00 | | Carnauba Wax | Lubricant |
| | | | | and |
| | | | | binder |
| | | | PART II Direct | |
| | | | Compression | |
| 12.00% | 30.00 | | HPMC (Benecel E5) | Glidant |
| 25.20% | 63.00 | | Mannitol | Binder |
| 0.80% | 2.00 | | Acesulfame Potassium | Sweetener |
| 2.00% | 5.00 | | Sodium Stearyl | Lubricant |
| | | | Fumarate (Alubra) | |
| 8.00% | 20.00 | | Aerosil 200 | Glidant |
| 8.00% | 20.00 | | Sodium | Disintegrant |
| | | | Crosscarmeliose | |
| 100% | 250.00 | 10.0 | | |

The solid formulations as exemplified in Tables 3 and 4 can be formulated as sublingual tablets containing THC and CBD combination and administered in therapeutically amounts to MM patients.

### REFERENCES

1. Avet-Loiseau H, Attal M, Moreau P, Charbormel C, Garban F, Hulin C, Leyvraz S, Michallet M, Yakoub-Agha 1, Garderet L et al: Genetic abnormalities and survival in multiple myeloma: the experience of the Intergroupe Francophone du Myelome. Blood 2007, 109(8):3489-3495.
2. Melton L J, Kyle R A, Achenbach S J et al (2005) Fracture risk with multiple myeloma: a population-based study. J Bone Miner Res 20:487-493
3. Kumar S K, Rajkumar S V, Dispenzieri A, Lacy M Q, Hayman S R, Buadi F K, Zeldenrust S R, Dingli D, Russell S J, Lust J A et al: improved survival in multiple myeloma and the impact of novel therapies. Blood 2008, 111(5):2516-2520.
4. Rajkumar S V: Treatment of multiple myeloma. Nature reviews Clinical oncology 2011, 8(8):479-491.
5. Bandana Chakravartil,*, Janani Ravi2,* and Ramesh K. Ganju2. Cannabinoids as therapeutic agents in cancer: current status and future implications Oncotarget, (2014). Vol. 5, No. 15
6. A. C. Howlett, F. Barth, T. I. Bonner, G. Cabral, P. Casellas, W. A. Devane, C. C. Felder, M. Herkenham, K. Mackie, B. R. Martin, R. Mechoulam, R. G. Pertwee International Union of Pharmacology. XXVII. Classification of cannabinoid receptors Pharmacol. Rev., 54 (2002), pp. 161-202.
7. CB2 Chemical Agents for Multiple Meyloma (M M) Intervention. Project collaborators: Xiang-Qun (Sean) Xie (PI, Dept. Pharmaceutical Sciences), D. Roodman (UPMC), Julie Roodman (UPMC), and Jurg Gertsch (University of Bern, Switzerland).
8. Maria Beatrice Morelli1, Massimo Offidani, Francesco Alesiani, Giancarlo Discepoli, Sonia Attilio Olivieri, Matteo Santoni, Giorgio Santoni, Pietro Leoni and Massimo Nabissi. The effects of cannabidiol and its synergism with bortezomib in multiple myeloma cell lines. A role for transient receptor potential vanilloid type-2. Int. J. Cancer. (2014) 134, 2534-2546.
9. Esteve F R, Roodman G D: Pathophysiology of myeloma bone disease. Best practice & research Clinical haematology 2007, 20(4):613-624.
10. Giuliani N, Rizzoli V, Roodman G D: Multiple myeloma bone disease: Pathophysiology of osteoblast inhibition. Blood 2006, 108(13):3992-3996.
11. Epstein J, Walker Myeloma and bone disease: “the dangerous tango”. Clinical advances in hematology & oncology: H&O 2006, 4(4):300-306.
12. Oshima T, Abe M, Asano J, Hara T, Kitazoe K, Sekimoto E, Tanaka Y, Shibata H, Hashimoto T, Ozaki S et al: Myeloma cells suppress bone formation by secreting a soluble Wnt inhibitor, sFRP-2. Blood 2005, 106(9):3160-3165.

1. A method for treatment of multiple myeloma in a subject, comprising the steps of:
(i) isolating multiple myeloma cells form the subject;
(ii) contacting the isolated multiple myeloma cells with different ratios of a first cannabis extract comprising cannabidiol (CBD) and a second cannabis extract comprising tetrahydrocannabinol (THC);
(iii) monitoring the cytotoxic effect of the different CBD:THC extracts ratios on the isolated multiple myeloma cells;
(iv) determining a CBD:THC extracts ratio that provides at least 50% inhibition of the multiple myeloma cells in vitro; and
(v) providing to the subject a therapeutically effective amount of a cannabis extract comprising CBD:THC in a ratio determined in (iv), thereby treating multiple myeloma in the subject.

2. The method of claim 1, wherein the first cannabis extract comprises from about 2% (wt.) to about 20% (wt.) CBD.

3. The method of claim 1, wherein the second cannabis extract comprises from about 0% (wt.) to about 20% (wt.) THC.

4. The method of claim 1, for personalizing a dose regime of a cannabis extract comprising CBD and THC, effective in treating multiple myeloma in the subject.

5. The method of claim 1, wherein the CBD:THC extracts ratio determined in step (iv) confers an additive effect with respect to inhibition of the multiple myeloma cells relative to the effect conferred by the corresponding CBD and THC extracts administered separately.

6. The method of claim 1, wherein the CBD:THC extracts ratio determined in step (iv) confers a synergistic effect with respect to inhibition of the multiple myeloma cells relative to the effect conferred by the corresponding CBD and THC extracts administered separately.

7. The method of claim 1, wherein CBD and THC in the cannabis extract have a combination index (CI) value lower than 1 indicating synergism.

8. The method of claim 1, wherein CBD and THC in the cannabis extract have a combination index (CI) value of 1 indicating an additive effect.

9. The method of claim 1, wherein the cannabis extract comprising the determined CBD:THC ratio is provided to the subject by one or more routes of administration selected from the group consisting of intranasal, transdermal, intravenous, vaginal, sublingual, buccal and oral.

10. The method of claim 9, wherein the cannabis extract is provided so as to penetrate the mucosal barrier.

11. The method of claim 1, further comprising providing to the subject an additional multiple myeloma therapeutic agent selected from the group consisting of an alkylating agent, a corticosteroid, a proteasome inhibitor, an immunomodulatory drug, and any combination thereof.

12. The method of claim 11, wherein said additional multiple myeloma therapeutic agent is selected from the group consisting of bortezomib (BTZ), lenalidomide (LEN), dexamethasone (DEX), melphalan (MEL), mitoxantrone, doxorubicin, Bortezomib-cyclophosphamide-dexamethasone (VCD), bortezomib-thalidomide-dexamethasone (VTD) and any combination thereof.

13. The method of claim 11, for achieving higher clinical response rates than when the multiple myeloma therapeutic agent is provided without the CBD and THC.

14. The method of claim 1, for inhibiting conventional chemotherapy resistant multiple myeloma cells.

15. The method of claim 14, wherein the conventional chemotherapy comprises a multiple myeloma therapeutic agent selected from the group consisting of bortezomib (BTZ), lenalidomide (LEN), mitoxantrone, dexamethasone (DEX), melphalan (MEL), doxorubicin (DOM)), Bortezomib-cyclophosphamide-dexamethasone (VCD), bortezomib-thalidomide-dexamethasone (VTD) and any combination thereof.

16. The method of claim 1, wherein the cannabis extract comprising the determined CBD:THC ratio is provided to the subject in a sustained release dosage form or in a rapid release dosage form or in a combination thereof.

17. The method of claim 16, wherein the cannabis extract comprising the determined CBD:THC ratio is provided to the subject once, twice, three or four times through the day.

18. The method of claim 17, wherein the dosage of CBD provided is of up to 400 mg per day, and the dosage of THC provided is up to 400 mg per day.

19. The method of claim 18, wherein the dosage of CBD is in the range of from about 2 mg to about 400 mg per day, and the dosage of THC is in the range of from about 10 mg to about 400 mg per day.

20. The method of claim 1, wherein the first cannabis extract comprising CBD, the second cannabis extract comprising THC, or both, is derived from at least one cannabis plant.

21. The method of claim 20, wherein the at least one cannabis plant is a CBD rich strain.

22. The method of claim 20, wherein the at least one cannabis plant is a THC rich strain.
